(19) United States
(12) Patent Application Publication
(10) Pub. No.: US 2002/0068968 A1
(43) Pub. Date: Jun. 6, 2002

(54) Virtual stent making process based upon novel enhanced plate tectonics derived from endoluminal mapping

(76) Inventor: Thomas Hupp, Stuttgart (DE)

Correspondence Address: Edwards Lifesciences LLC, Law Dept., One Edwards Way, Irvine, CA, 92614, US

(21) Appl. No.: 09/930,514

(22) Filed: Aug. 15, 2001

Related U.S. Application Data
(63) Continuation-in-part of application No. PCT/US01/24656, filed on Aug. 4, 2001.
(60) Provisional application No. 60/227,070, filed on Aug. 22, 2000.

(30) Foreign Application Priority Data
Aug. 16, 2000 (DE) 10040630.0

(51) Int. Cl. A61F 2/06

(52) U.S. Cl. 623/1.15

(57) ABSTRACT

The present invention concerns a process for making a virtual stent for implantation into a body lumen. A particularly preferred embodiement is optmized for emplacement and indwelling in the internal carotid artery, with a lower end and an upper end wherein the radius decreases from lower end to upper end, and includes ‘trumpet-like’ or parabolic elements abutting facultative apertures. Generally, computer-aided-design derived stent has a tectonic structure in the form of angles and curvatures adapted to the course of a desired lumen. For example, the internal carotid artery whereby lower end in the region of the outlet of internal carotid artery is formed as an ovaloid recess provided in the region of the outlet of the external carotid artery.

### CROSS-REFERENCE TO RELATED APPLICATIONS

This application claims full Paris convention priority rights from DE10040630.0 filed Aug. 16, 2000, and the U.S. Provisional Patent Application Serial No. 60/227,070 filed Aug. 22, 2000 entitled STENT ZUR IMPLANTATION IN DIE HALSSCHLAGADER. Likewise, the instant application is a continuation in part of PCT/US01/24656, lodged Aug. 4, 2001, each of the same authored by the present inventor, with the latter including EDWARDS LIFESCIENCES LLC as co-applicant.

### BACKGROUND OF THE INVENTION

The present invention relates to novel apparatus and processes for maintaining patency of body lumens. In particular, the present invention supplies novel enhanced customized, optimized or otherwise individuated stents, useful for example in the carotid artery—wherein a generally conical/‘trumpet-like’/or parabolic shaped member having free ends—is emplaced into the native vessel to provide for enhanced blood flow.

Attention is called to the following European and PCT patent applications, publications, and United States Letters Patents, which are expressly incorporated herein by reference as defining the state of the art:

| US 2001/0004705; | EP1101456A1; | WO 99/44,540; |
|---|---|---|
| WO 98/53,764; | EP 0923,912 A2; | 6,248,129 B1; |
| 6,159,238; 6,106,548; | 5,938,697; and | WO 98/53759; |

Palmaz, J C. Molecular Approaches to Devices and Materials (ABSTRACT) ISES 2001; Sprague E A, Palmaz J C, Simon S, Watson A. Electrostatic Forces on the Surface of Metals as Measured by Atomic Force Microscopy. J Long-Term Effects Med Implants 2000; 10:111; Simon C, Palmaz J C and Sprague E A. Protein Interaction With Endovascular Prosthetic Surfaces. J Long-Term Effects Med Implants 2000;10(1-2): 127-141; each of which has been studied and differentiated from the instant teachings, while highlighting the longstanding need for the same.

### SETTING OF THE INVENTION

Whether the term “stent” is derived from Dr. Stent's mass or the old English verb “to stint” the meaning today is an inner support for a body lumen.

Plethoric problems relating to the stenting of anatomically challenging lumens, including bifurcated endoluminal regions, have been well documented (See, for example, the patents and publications referenced above and the publications listed below, each of which is expressly incorporated herein by reference as if fully set forth). However, global solutions remain pending.

Likewise, as techniques and devices having peripheral vascular applications evolve, the carotid arteries have become prominent as housing target stenotic lesions. This is further bolstered by the ongoing trend that extends patients' viability longer exposing greater number of patients to the need for carotid artery intervention at some point in their medical history.

The present inventor has had a review of the literature undertaken, and has discovered significant trends impacting the longstanding needs for novel enhanced carotid arterial therapies. While Carotid endarterectomy (CEA) is well established as a safe and effective procedure to treat significant disease of the carotid bifurcation, subgroups of patients who may not benefit from CEA have become known. (Sandager, G. Duplex Evaluation of the Extracranial Carotid Arteries Pre and Post Carotid Stent. [ABSTRACT] ISES 2001.) Balancing against the strong need for alternate therapies is the risk of embolic events feared in those patients having unstable (ulcerating) plaque morphologies. It is respectfully proposed that as a threshold issue, the ‘match’ between a strictly tubular stent and a truncating or intnerally tapered vessel anatomy must be reviewed.

Referring now to TABLE 1, styled RESULTS OF CAROTID STENTING IN THE LITERATURE 1996-2000 (appended hereto and expressly incorporated by reference herein) incorporates data which shows that rates of apoplexy, morbidity and mortality range from about 3% to about 10% for carotid stenting done to date. This makes comparison of the safety and efficacy of endovascular procedures difficult to compare with CEA. It is believed that in addition to prospective randomized studies in this area, a frank evaluation of whether conventionally tubular stents are appropriate in the carotids constitutues a longstanding need.

The present invention concerns a stent for implantation into the carotid artery according to a process whereby a lumen of a vessel is mapped to ensure compliance of the stent geometry and the lumen. Stents are endoprostheses in the form of grid supports, which are utilized at places of constriction in body vessels, in order to again produce undisturbed blood flow, inter alia. In some cases they may serve for widening the constriction, so that the inner diameter or the inner lumen of the affected vessel is again brought to the usual width, and further for the stabilization of the vessel wall. Conventional stents are formed as tubes or hollow cylinders and are comprised of metal or plastic latticework in various forms.

One generally distinguishes between balloon-expandable stents, which are brought into their final form by means of a balloon catheter, and self-expanding stents comprised of a material with memory effect, which are automatically converted into their final form by heating in the body. The possible applications of these stents in vessels of great variability are determined by the various radial diameters, lengths, and flexibility properties.

Likewise, combining self-expanding and balloon expandable stents and stent grafts is contemplated as within the scope of the instant teachings.

To date, it is respectfully proposed that it has turned out in practice that conventional stents are only poorly suitable or even not suitable for implantation in the carotid artery for treatment of a constriction (stenosis) of the carotid artery (Arteria carotis). The reason for this is the special configuration of the carotid artery.

The conclusions of the present inventor are based on empirical observations including the following. The carotid artery has a division of the vessel (so-called bifurcation) at which the actual common carotid artery (Arteria carotis communis) divides into an internal carotid artery (Arteria carotis interna) and an external carotid artery (Arteria carotis externa). At such bifurcation, one or both vessel outlets are displaced by the wall of the known stents.

The vessel form usually does not correspond to a tube with a constant internal diameter, but is ‘trumpet’ or cone-shaped/conical viewed in an anatomically correct manner, thus continually tapering in one direction. This tapering is in fact negligible in the case of the larger vessels, but is particularly clearly pronounced in the carotid artery and is of great importance with respect to the precision of fit of the stent.

There is a clear reduction in the radius in the distal vessel course, particularly in conjunction with the bifurcation of the common carotid artery. Conventional stents in tube form thus fit poorly, since they have either a diameter that is too small at one end or too large at the other end.

The smaller the lumen of the vessel, i.e., the smaller its internal diameter, the more probable that there is a curve-shaped course of the vessel in adaptation to individual anatomical features. This curving is also increased to a particular extent in the case of the carotid artery. The curve-shaped region is also associated with bending segments in the carotid artery, whereby the direction of principal blood flow varies in all three dimensions in space.

Conventional stents bend sharply or buckle in the region of these bendings, whereby the stents themselves become constricted and hinder the flow of blood. A brief perusal of the prior art underscores these conclusions. A radially expandable stent is disclosed in EP 0 884,028 A1 for implantation in a body vessel in the region of a vessel bifurcation. This stent in fact has an enlarged radial opening in the region of the vessel bifurcation, but is formed as a simple tube and is thus not suitable for implantation in the carotid artery.

In sum, having practiced surgical intervention within the human vasculature for some time, and observed the shortcomings of the prior art devices, the present inventor was compelled to conclude that the carotid artery has different needs in terms of stenting devices. To these ends, the present inventor has used geometric measurements of human carotid bifurcations to generate parameters to optimize a stent for carotid use, in addition to comparing the angular variability of diseased (“ASKL”) carotid bifurcation segments, with those without arteriosclerosis (“Ohne ASKL”). Table 2 summarizes these results in a graphic form, and is herewith expressly incorporated by reference as appended hereto.

### OBJECTIVES AND SUMMARY OF THE INVENTION

Accordingly, among the objectives of the present invention are to provide a carotid stent having features derived from geometric measurements of a series of appropriate vessel lumens, and to teach how to do the same by a process.

Another object of the present invention is to provide at least a set of design features derived from an empirically determined set of radius ratios and/or an algorithm defining the relationships among the internal carotid artery, external carotid artery and the common carotid artery useful in modeling a desired stent geometry.

According to the teachings of the present invention stents for implantation into the internal carotid artery are disclosed, with a lower end and an upper end, wherein the radius from lower end to upper end decreases, characterized by the fact that stent has a tectonic structure in the form of angles and curvatures adapted to the course of the internal carotid artery and that in the region of the outlet of internal carotid artery, a lower end is formed as an ovaloid opening.

Likewise, there is taught a stent for implantation in the internal carotid artery, with a lower end and an upper end, wherein the radius decreases from lower end to upper end, characterized by the fact that a stent having a tectonic structure in the form of angles and curvatures is adapted to the course of internal carotid artery, with a lower end projecting into the common carotid artery and having an ovaloid recess provided in the region of the outlet of the external carotid artery.

The foregoing are achieved in novel enhanced apparatus for implantation into the carotid artery formed by a process using luminal mapping and products by the process, as set forth in the claims appended hereto and disclosed herein to one having ordinary skill in the art.

According to a further feature of the present invention there is provided a stenting apparatus configured to correspond to the endoluminal surface of a carotid artery having a tectonic structure in the form of angles and curvatures following the course of a patient's internal carotid artery and having an ovaloid aperture communicating with the external carotid artery, the improvement which comprises a trumpet-like tapered section from proximal to distal ends, whereby the spatial orientation of the stent defines a substantially hyperbolic section adjacent said ovaloid aperture.

According to yet a still further embodiment of the present invention there is provided a process for generating a novel enhanced stenting device, comprising the steps of; targeting a luminal surface to be mapped, capturing a non-contact picture of the surface data of the luminal surface to be mapped, generating a multiplicity of three-dimensional measuring points, arraying said multiplicity of three-dimensional measuring points within a predetermined lattice structure defining a tectonic structure in the form of angles and curvatures adapted to the course of the mapped luminal surface; providing individuated or otherwise customized sections of geometric scaffolding structure corresponding to the portions arrayed in the lattice by three-dimensional computer modeling to make a virtual stent.

Briefly stated, a stent according to the invention is provided for the region of the branching of the common carotid artery which has an anatomically correct adaptive from, which takes into consideration the special features in the region of the bifurcation of the common carotid artery and of the course of the proximal part of the internal carotid artery, i.e., found directly at the bifurcation of the common carotid artery.

### BRIEF DESCRIPTION OF THE FIGURES ILLUSTRATING THE INVENTION

The file of this patent contains at least one color photograph. Copies of this patent with the photographs will be provided by the Patent and Trademark Office upon request and payment of the necessary fee.

The present invention will be further explained on the basis of the attached figures, serving to be illustrative rather than limiting of the teachings of the present invention. Schematic depictions are herewith offered for consideration, wherein:

FIG. 1 shows a schematic, perspective representation (not to scale) of the carotid artery in the region of the bifurcation;

FIG. 2 shows a schematic, perspective representation (not to scale) of a first example of embodiment of a stent according to the invention in the carotid artery;

FIGS. 3A to 3D show schematic, perspective representations (not to scale) of a second example of embodiment of a stent according to the invention in the carotid artery from various perspectives;

FIG. 4 is a schematic view of a virtual stent according to an embodiment of the process of the present invention, the same being generated by plotting at least about 500,000 data points within a carotid application;

FIG. 5 is another schematic view of another virtual stent according to yet a still further embodiment of the process of the present invention and products generated thereby, the same being generated by plotting at least about 500,000 data points within a carotid application;

FIG. 6 shows a schematic view of a stent made from a cast made from a set of data points from carotid arteries modeled on a computer-aided design system whereby an inner-lining or customized stent is made according to yet a still further embodiment of the process of the present invention and products generated thereby;

FIG. 7 is a photographic image of a cast made from a harvested carotid artery according to the teachings of the present invention and the process thereunder;

FIG. 8 shows a digital photographic image of a plan view of an embodiment schematically mapping a virtual stent with computer-aided-design for an embodiment as shown in FIG. 3 above, whereby a cast is digitally overlaid;

FIG. 9 is digital photographic image comprising a virtual stent according to an embodiment of the process of the present invention, the same being generated by plotting at least about 500,000 data points within a carotid application;

FIG. 10 likewise shows a digital photographic image of a rotated and carotid bifurcation orientated view of an embodiment schematically mapping a virtual stent with computer-aided-design with respect to an embodiment as shown in FIG. 3 above;

FIG. 11 shows a (stent scaffolding geometry free) view of a ‘trumpet-like’ portion adjacent to the ovaloid opening in a first embodiment of the present invention, including the three ordinal planes (X, Y, and Z) as reference points and parabolic curvilinear markers (A, B) defining the peripheral portions of the involved stent;

FIG. 12 shows a (stent scaffolding geometry free) view of a ‘trumpet-like’ portion adjacent to the ovaloid opening in a second embodiment of the present invention, including the three ordinal planes (X, Y, and Z) as reference points and parabolic curvilinear markers (A, B) defining the peripheral portions of the involved stent; and,

FIG. 13 shows schematically the location of the view shown in FIG. 12 relative to a second embodiment of the instant teachings as illustrated in the FIG. 3 series above.

### DETAILED DESCRIPTION OF PREFERRED EMBODIMENTS OF THE PRESENT INVENTION

It is believed that current views of the medical literature suggest that endovascular therapies for extracranial carotid stenoses present a potentially valid alternative to carotid endarteractomy, or thrombectomy-based open surgical procedures. However, a mismatch of the anatomy of the carotid bifurcation and internal carotid anatomy and conventional tubular stents is offered for consideration as a primary issue requiring improvement.

The present inventor has discovered why the conventional tubular stent does not fit into the carotid artery, particularly the bifurcated region within human carotid arteries. Empirical studies with casts made from harvested human carotids have shown that curvature in the proximate internal carotid artery [ICA] the differences in cross-sectional area along the course of the ICA and variations in angles found at the bifurcation result in the need for a differently shaped stenting device. A computer-aided-design (CAD) system enables the instant teachings to be actualized in a novel enhanced carotid stenting device according to the instant teachings.

The stent according to the invention for the region of the branching of the common carotid artery has an anatomically correct adaptive from, which takes into consideration the special features in the region of the bifurcation of the common carotid artery and of the course of the proximal part of the internal carotid artery, i.e., found directly at the bifurcation of the common carotid artery.

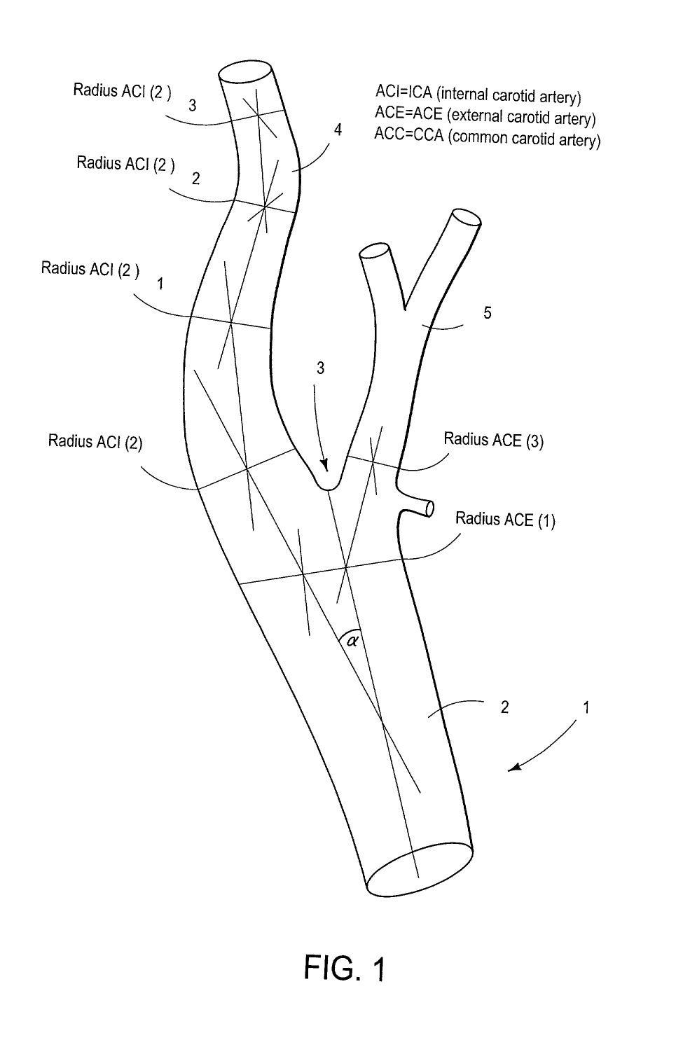

These special features are schematically shown in FIG. 1. The region of the carotid artery 1 that is shown includes as the principal branch the upper region of the common carotid artery [ACC] 2, the vessel forking or bifurcation 3, and as secondary branches, the lower regions of the internal carotid artery (Arteria carotis interna) ICA 4, and the external carotid artery (Arteria carotis extema ) [ACE].

It is seen that the vessel radius of the CCA (1) is greatest in the region of common carotid artery 2. The internal carotid artery 4 narrows proceeding from the vessel bifurcation 3 so much so that the vessel radii ICA (2), ICA (2₁), ICA (2₂), ICA (2₃) decrease continually. Also, the vessel radius of the external carotid artery 5 ECA (3) is smaller than that of the common carotid artery 2 and is also smaller than the vessel radius ICA (2) of internal carotid artery 4.

Finally, the angle a (the outlet angle of the internal carotid artery) varies; it is different for each person. It has also been demonstrated that the internal carotid artery 4 practically never pursues a linear course. The internal carotid artery 4 curves to a great extent in all three spatial directions.

The constrictions that occur most frequently (stenosis) of carotid artery 1 are found in the upper region of common carotid artery 2 and in the lower region of internal carotid artery 4. The stent must be placed in the region of the internal carotid artery and of the common carotid artery in these cases. The outlet of the external carotid artery 5 in the region of the vessel bifurcation 3 in these cases is sealed off by the wall of a conventional stent, so that blood can no longer flow through the external carotid artery 5 or at least the blood flow is severely adversely affected when blood flows into the external carotid artery through the grid network.

Likewise, conventional stents can be placed exactly only poorly with the size provided, due to the variable vessel diameter and the curvature of the internal carotid artery. In sum, currently available stents have some efficacy with respect to stenting of the outlet of the ICA and/or ACE branches, but that is where conventional technology ends. To address the bifurcation raises issues that are not within the ambit of conventional teachings.

There are those who are of the view the loss of the ACE and its branches is trivial, but the fact remains that the external branches provide a possible collateral flow to the intracranial vascular circulation, placing a premium upon their safe preservation.

Likewise, selection and emplacement of stenting apparatus is challenging owing to the tremendous variation in sizing between the tranverse diameter or the ACC and ACI in complex lesions affecting the carotid bifurcation. Table 3, is offered for consideration in these regards, the same being appended to the instant specification and expressly incorporated herein by reference as showing one set of area calculations derived from casts made as shown in FIG. 1, as explained further below.

With conventional tubular stents, the involved technical manoeuvres and dilatation catheters required to safely attach stents to artery walls around the bifurcation are truly daunting to the average practitioner. Likewise, it is often the case that relatively ‘fresh’ arteriosclerotic material is found local to stenotic lesions in the carotid bifurcation area.

The present inventor respectfully proposes that tubular stents currently in general use are not anatomically suitable based upon a series of geometric measurements of human carotids. Likewise a novel enhanced approach is offered for consideration, in part on the basis of the works discussed below.

### EXAMPLES

### Materials & Methods

118 carotid bifurcation samples were harvested from autopsies and respective packets of individuated data points arrayed in a specialized database (EXCEL®, Microsoft Corporation, Redmond, Wash. State, U.S.A.) including relevant information from the autopsy register about the cause of death and underlying illnesses in addition to sample side (right versus left) and all height and weight data.

Casts were prepared from the harvested vessels by suspending them, suitabley prepared at a suspension facility, in the direction of flow. The vessels were then drained with a fast-hardening plastic (PALADUR R, Heraeus Kulzer GmbH, Wehrheim, Germany), and cured for ⅓ of an hour at approximately 23 degrees Celcius. The resulting hardened cast preparations were compared with the harvested vessels, which were preserved in formalin, cataloged and anterior/posterior projections documented photographically.

Calliper gauge measurements were taken (MITUTOYO® Digital Calliper, Japan) at a resolution of 0.01 mm. Maximum and minimum diameters were measured first in two dimensions, at defined measuring points on the ACC (Dc1min/Dcmax/Dc2min/Dc2max), ACI (Di1min/Di1Max/Di2Min/Di2max and the ACE (Demin/Demax) as well as the carotid bulbus (Cbmin/Cbmax). Cross-sectional area measurement followed, and were based upon prior accepted sites for measuring points known to those skilled in the art.

Two-dimensional measurements of the cast preparations were done with a projection gauge (WERTH RECORD 400 measurement projection gauge) that were then magnified by 10× upon projection onto a screen were the same were traced and then measured.

Referring still to FIG. 1, diameters of the ACC, ACI and ACE were recorded at defined points by geometric construction, the angles αi and αe were derived for mainstream direction of the ACC to the ACI or ACE. Likewise, it was possible to calculate the curvature in the proximal ACI (ρ/Di1).

Three-dimensional reconstruction of a virtual prototype for an anatomically formed carotid stent was thus enabled for each selected cast preparation. This was done by selecting a cast preparation, capturing a non-contact picture of the surface data with a laser scanner (HYSCAN 45 C 3D Laser-Scan-Head Model 50, Hymarc®, Germany) at a time interval of at least about 2 hours. Approximately 574,893 three-dimensional measuring points (for example, on that cast which was designated as 140/98L) were generated by computer-aided-design (SURFACER V 8.0 software, by Imageware®) with the assistance of the Fraunhof Institute for Production Engineering and Automation (IPA) in Stuttgart, Germany.

Statistical evaluation was likewise undertaken on the basis of the anamnestic data for each individual, including mean value, standard deviation, median, 1^{st} and 3^{rd} quartile and maximum and minimum parameters. The statistical evaluation system SAS was employed (SAS Institute Inc., Cary, N.C., U.S.A.).

The novel enhanced and optimized carotid stenting devices of the present invention are based in part on conclusions that median curvatures along the proximal ACI between measuring points I1 and I2 significantly differ from zero, or that the course of the proximal ACI cannot be considered to be rectilinear. Table 7 is offered for consideration in these regards, the same being appended to the instant specification and expressly incorporated herein by reference as demonstrative of this geometric conclusion.

Likewise, there is a significant difference in a cross-sectional areas comparison between the first measuring point (AI1) on the on the ACI, immediatlye at the outlet, and the second measuring point (AI2). Table 6 is offered for consideration in these regards, the same being appended to the instant specification and expressly incorporated herein by reference as demonstrative of this geometric conclusion about the carotid lumens reviewed.

Further, among the various cross-sectional areas reviewed, only minor differences could be detected between preparations from patients with and without artiosclerosis. Table 4 is offered for consideration in these regards, the same being appended to the instant specification and expressly incorporated herein by reference as demonstrative of this geometric conclusion about the carotid lumens reviewed.

Similarly, only minor differences were noted between male and female vasculature. Table 5 is offered for consideration in these regards, the same being appended to the instant specification and expressly incorporated herein by reference as demonstrative of this geometric conclusion about the carotid lumens reviewed.

Nor were Body Mass Index (BMI) data and cross sectional data found to have any statistical relationship. Table 11 is offered for consideration in these regards, the same being appended to the instant specification and expressly incorporated herein by reference as demonstrative of this geometric conclusion about the carotid lumens reviewed.

It was found that the three dimensional modeling was also helpful in designing the virtual stent to be placed in the ACC in the bulbus area, the ACI over the outlet area and, preferably, over the recess of an outlet opening for the ACE outlet. As is known to artisans, the involved Surfacer® (V8) software enables saved geometrical data of such a virtual prototype to be converted into manufacturing data (NC data) and then rapidly prototyped. Those skilled would also be able to readily substitute metals, plastics, shape-memory alloys and the like for stent materials having a desired characteristic.

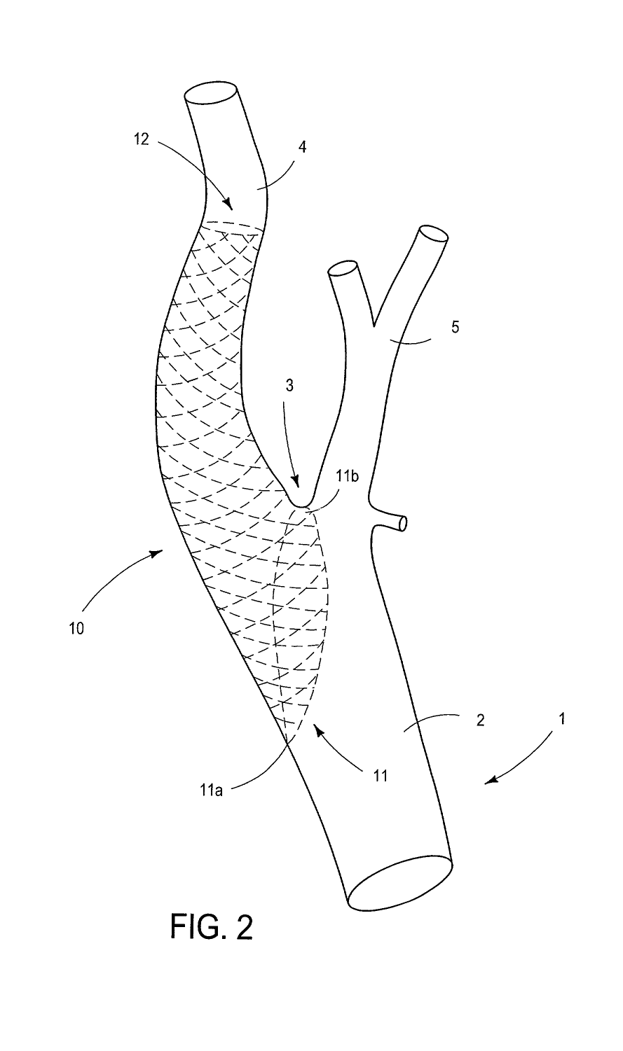

Referring now to FIG. 2, a first example of an embodiment of a stent 10 according to the invention for implantation in the internal carotid artery is shown. Stent 10 has a lower end or an inlet 11 and an upper end 12. Inlet 11 is found in the region of vessel bifurcation 3 and is shaped in ovaloid form in order not to cover the outlet of the external carotid artery 5.

Inlet 11 is obliquely sectioned due to the ovaloid structure and partially projects into the common carotid artery by its longer end 11a and thus supports the vessel wall in the region of the outlet of the internal carotid artery at the vessel bifurcation 3. The shorter end 11b of inlet 11 supports the internal carotid artery 4 directly at vessel bifurcation 3. In this away, the outlet of internal carotid 4 is securely kept open. Stent 10 is also shaped like a cone, wherein the radial diameter varies in the longitudinal course and becomes smaller proceeding from inlet 11 upper end 12, so that it is adapted to the course of internal carotid artery 4. Likewise, the geometry of stent 10 can be characterized as roughly parabolic, making reference to the three ordinal planes which are shown at each radius-based juncture where measurements are taken. The conical, parabolic, or ‘trumpet-shaped’ nature of stent 10 is noted as distinct from those generally tubular stents which are known and used conventionally.

Stent 10 has a tectonic structure, i.e., angles and curvatures in three-dimensional space adapted to the course of internal carotid artery 4. Stent 10 according to the invention is characterized by an anatomically corrected adaptive form, as further defined below within the claims that are appended hereto.

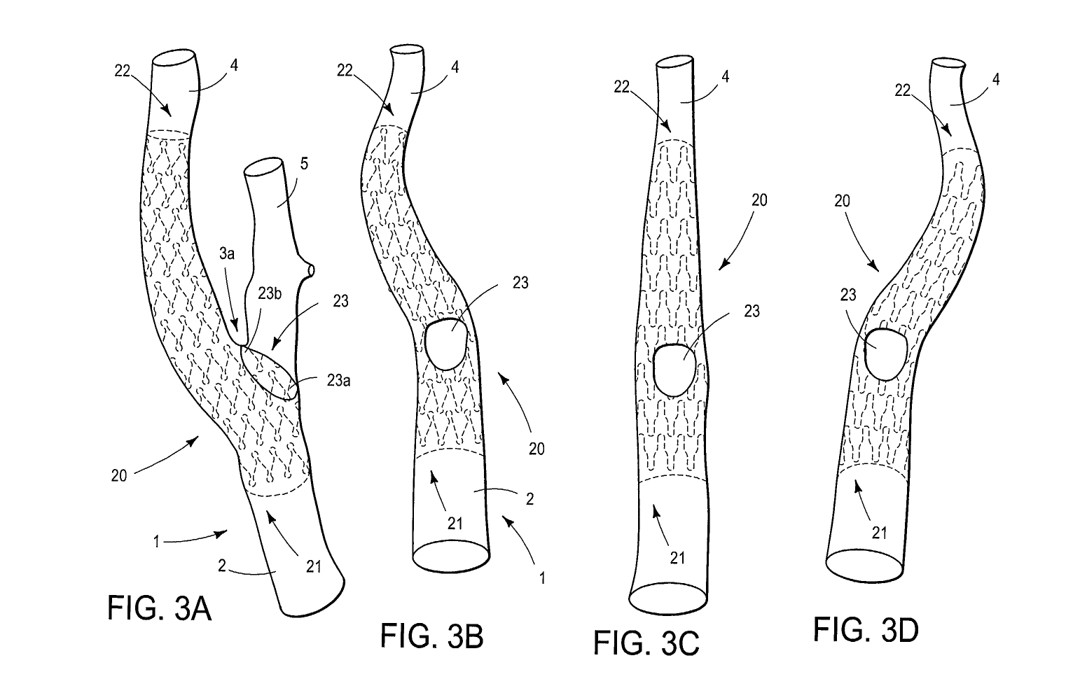

FIGS. 3a to 3d show another example of an embodiment of a stent 20 according to the invention. Stent 20 also has a lower end 21 and an upper end 22. Lower end 21, however, is now found within the common carotid artery. In the region of vessel bifurcation 3, an ovaloid recess 23 is now provided, which lies precisely at the outlet of external carotid artery 5. Please note that, as shown, it is possible to use patterns, portions, or desired aspects of any known or developed stent geometry or scaffoldijng structure to satisfy the desired tectonic structure.

Due to the ovaloid formation, recess 23 is also obliquely sectioned and now slightly projects into external carotid artery 5 by its longer end 23a. Shorter end 23b of recess 23 also supports internal carotid artery 4 directly at vessel bifurcation 3. This configuration has the advantage that the restraining or radial forces of stent 20, which keep open the outlet of internal carotid artery 4 at vessel bifurcation 3, already act at the upper end of the common carotid artery and do not excessively load the vessel walls in the region of the vessel bifurcation.

Stent 20 is also shaped like a cone, as is stent 10, wherein the radial diameter varies in the longitudinal course and becomes smaller from lower end 21 to upper end 22, so that it is adapted to the course of internal carotid artery. Stent 20 likewise has a tectonic structure, i.e., it has angles and curvatures in three-dimensional space adapted to the course of internal carotid artery 4, or it is “trumpet-shaped” or roughly parabolic. Stent 20 according to the invention is characterized by an anatomically correct adaptive form, whereby it is differentiable from the conventional tubular stenting devices which make up the majority of the prior art disclosures.

Stent 10 or 20 is comprised of a grid network, which can be formed of metal and/or plastic. The material may also be bioresorbable. The grid network may be introduced in the desired from by a balloon catheter, or it may have a memory effect, so that it is converted to the desired from automatically by the action of body heat.

Due to the tectonic structure of the grid frame, stent 10, 20 can reconstruct the curve-shaped course of the internal carotid artery in three-dimensional space. Bending at a sharp angle is thus avoided. This is shown in FIGS. 3a to 3d, which show stent 20 from a total of four different perspectives, wherein the three-dimensional curvature of the internal carotid artery 4, which follows stent 20, can be seen. Tables 8, 9 & 10 are offered for consideration in these regards, the same being appended to the instant specification and expressly incorporated herein by reference as demonstrative of this geometric conclusion about the carotid lumens reviewed.

The adaptation capacity of stent 10, 20 according to the invention can be clearly recognized relative to the potential alignment of the carotid artery in three-dimensional space, of the basis of these different perspectives. The lattice frame can be stamped from a tube or produced from wire, for example, bent, braided, knitted, or the like. The three-dimensional tectonic structure of stent 10, 20 is formed in production. Production may be conducted to yield various prepared sizes or individually adapted to the individual requirement. Implantation is conducted endoluminally.

Generally, as set forth above, a carotid bifurcation consists of a main branch, the ACC, which divides (but is not bifurcated) into two branches, the ACI and ACE. The ACI widens into the proximal part and is ‘trumpet-like’ and has a greater radius than that of the ACE. The final section of the ACC in the bifurcation and immediate ACI outlet may also be described anatomically as the carotid bulbus. The course of the ACC remains generally rectilinear having a relatively constant diameter without vascular outlets along its length. The ACI is partially linear, but conical or parabolic in the in the immediate outlet area. This means that the ACI is both tubular and conical/parabolic, but mainly curved. The ACE has numerous side branches along its length, the first of which is the superior thyroid artery. The superior thyroid artery generally originates partly from the bulbus or up to 2 cm distally therefrom.

Outside of the teachings of the instant invention, the present inventor is unaware of any studies that have developed aspects of the geometry of the carotid bifurcation with the exception of the use of angiography and related techniques. To these ends, the literature ranges from about 5.8 mm to about 8.6 mm for the median value of the diameter of the ACC, while the instant teachings provide 5.51 mm to 6.86 mm at C1 and C2. It is noted that with cadavers, “shrinkage” is likely to occur, and the same is expected with all of the instant measurements.

Turning to the second measuring point, C2, on the ACC, the median diameter of 5.16 mm DC2min and of 6.36 mm for DC1max were recorded by the present inventor. The proximal ACI (next to the outlet) yielded values of between about 5.62 mm and 6.49 mm. For the distal measuring point in the ACI, the I2, the median values ranged from at least about 4.04 mm to about 4.69 mm. Table 8 summarizes this set of relationships, and has been previously offered herein for consideration.

In terms of the bifurcation angles themselves, the instant teachings averaged 44.85 degrees. A significant difference between the present invention and all of the literature to date are that cross-sectional determinations on all recorded measurements (ACC, ACI, ACE—see Tables 3 & 4) were utilized (as opposed to solely diametric numbers) showing the curvature along the proximal ACI and the ACI's narrowing surface.

As discussed, the materials for stenting devices according to the instant teachings are known to those skilled in the art. However, combinations of self-expanding and balloon-expandable stents are unique to the instant teachings for use in the carotid artery, for example. Likewise, stainless steels, shape memory alloys, cobalt-based alloys and bioabsorbabable resins such as PLLA, PDLA, and PGA (PURAC America, Lincolnshire, Ill., USA) are contemplated. Similary, bioabsorbable polymers, silicones, corethanes and other known materials are effectively employed within the scope of the instant teachings.

In terms of the fluid dynamics relating to stenosis, more work is needed but there are several significant aspects of the instant teachings impacting upon the same. With the unique three-dimensional reconstruction of a virtual prototype for an anatomically formed carotid stent that was enabled for each selected cast preparation, new ground was broken.

It is respectfully proposed that now that a more accurate map of the carotid aterial lumen has been charted the use of 3-Dimensional colour duplex sonography, MR angiography and/or spiral CT scans of the carotid may be used with a three dimensional reconstruction to optimize stenting devices.

However, having harvested carotid arteries from cadavers, corrections need to be made for shrinkage of the vessels prior to generation of the appropriate software for general carotid modeling. It is likewise important to Note that the basic process involved selecting a cast preparation, capturing a non-contact picture of the surface data with a laser scanner (HYSCAN 45 C 3D Laser-Scan-Head Model 50, Hymarc®, Germany) at a time interval of at least about 2 hours. Approximately 574,893 three-dimensional measuring points were utilized by the present inventor in making the instant virtual stent, although different numbers of data points are differentially employed based on the application for the involved stenting device.

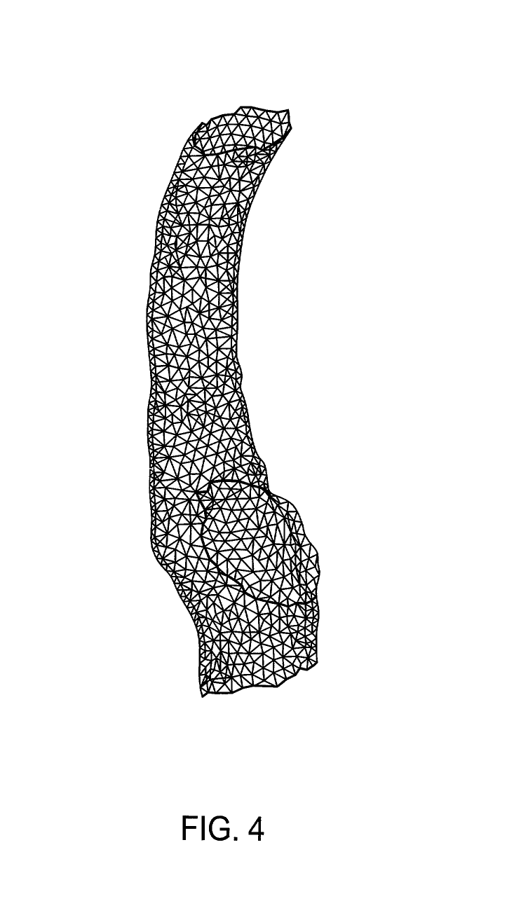

Referring now to FIG. 4-13, a series illustrating the practice of the process covered by the claims appended hereto is offered for consideration. Turning first to FIG. 4, a schematic view of a virtual stent according to an embodiment of the process of the present invention is shown, roughly geometrically conforming to the architrecture is FIGS. 3. In this case, the same was generated by plotting at least about 500,000 data points within a carotid application as discussed above.

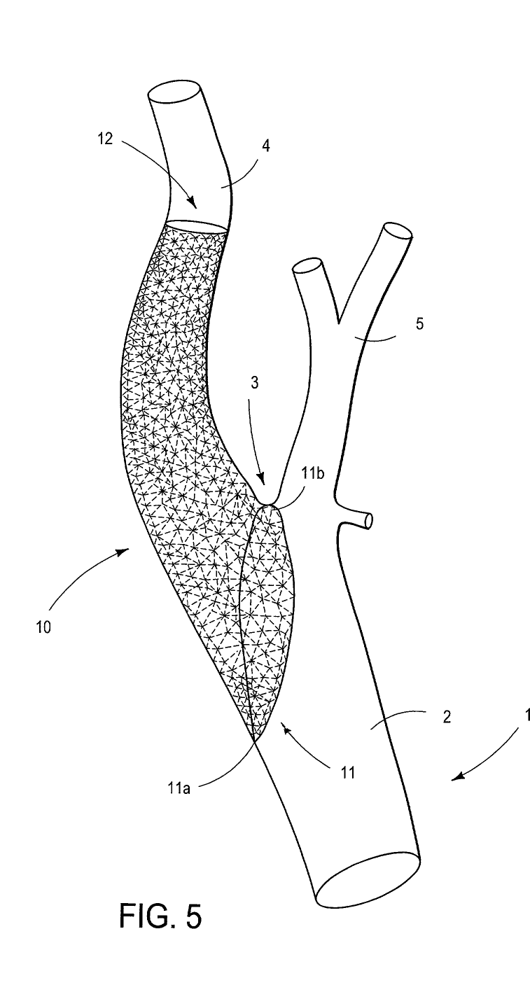

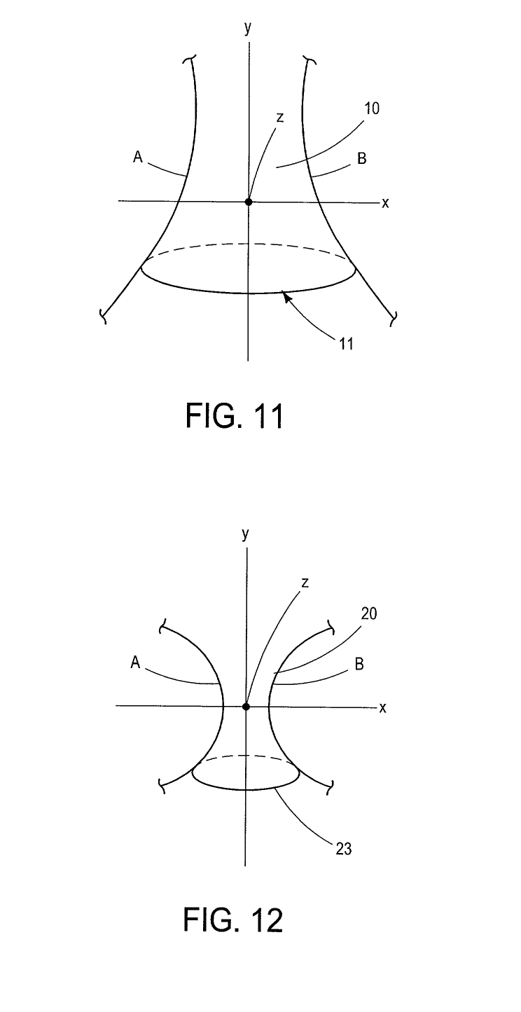

Turning to FIG. 5, another schematic view of another virtual stent according to yet a still further embodiment of the process of the present invention and products generated thereby is shown the same being generated by plotting at least about 500,000 data points within a carotid application. Note that the ‘trumpet-like’ orientation from 12 to 11, ends with a parabolic curved section (as further shown in detail at FIG. 11, below.)

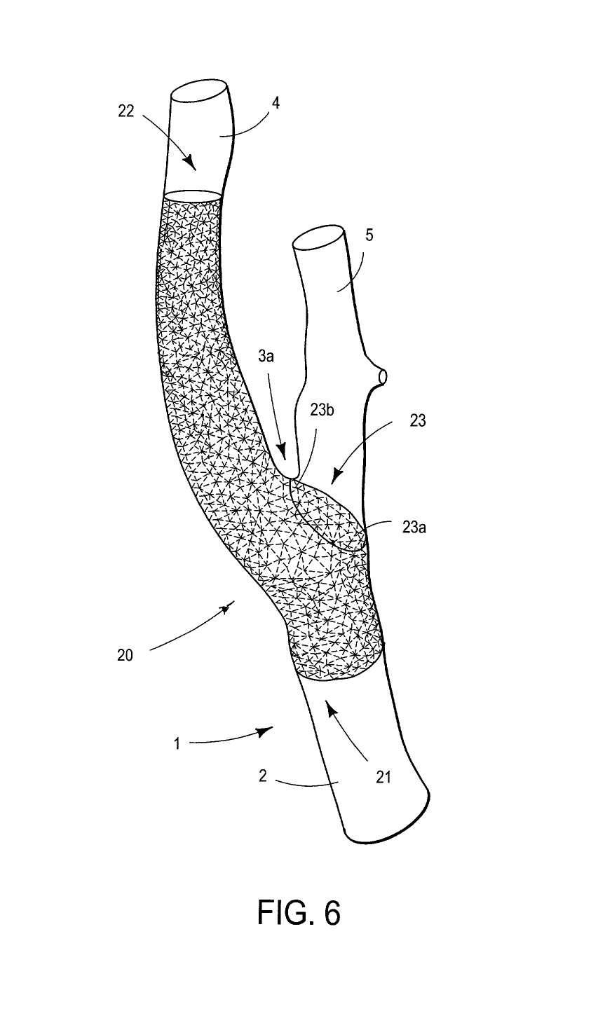

FIG. 6 shows a schematic view of a second embodiment of stent 20 made from cast made from a set of data points from carotid arteries modeled on a computer-aided design system. The reference designators denote the same elements.

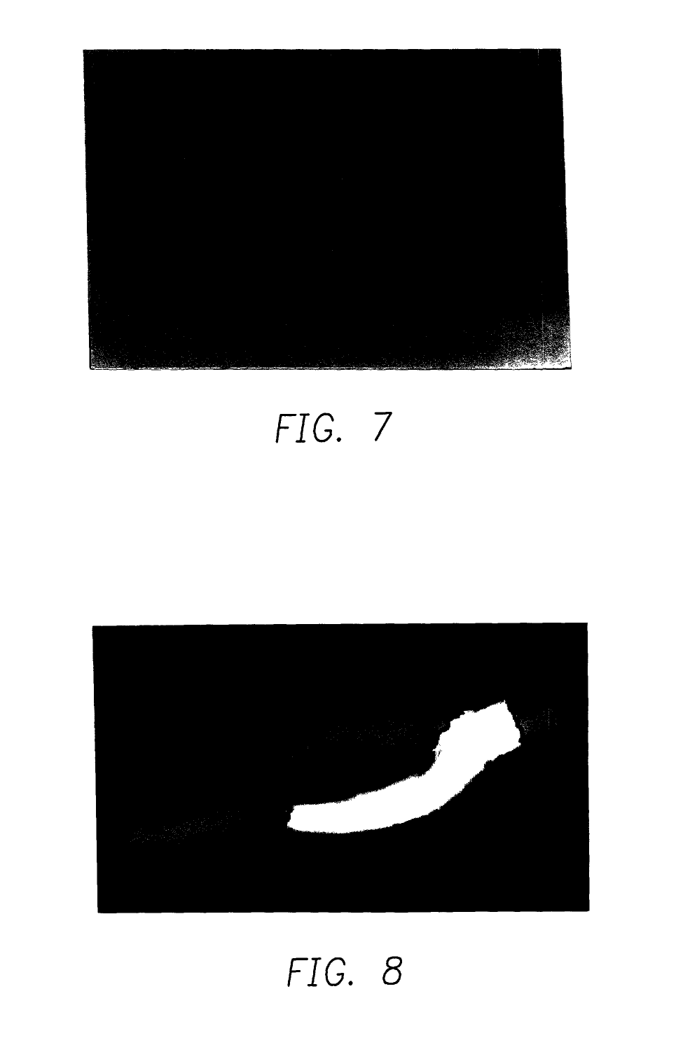

Turning now to FIG. 7 a digital photographic image comprising a virtual stent according to an embodiment of the process of the present invention, the same being generated by plotting at least about 500,000 data points within a carotid application is shown which corresponds to the schematic in FIG. 6.

FIG. 8 likewise is a digital photographic image of a plan view of an embodiment schematically mapping a virtual stent with computer-aided-design with respect to an embodiment as shown in FIG. 3 above. It is noted that applicant contemplates the use of any number of different stent scaffolding materials or patterns as discussed above.

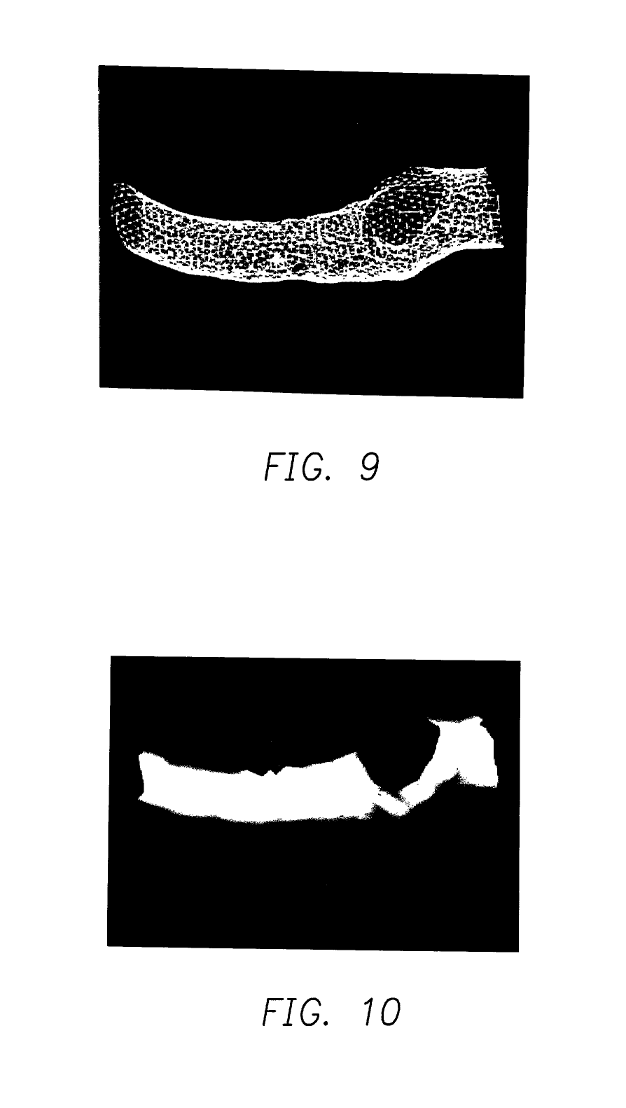

FIG. 9 is also a digital photographic image of a rotated and carotid bifurcation orientated view of an embodiment schematically mapping a virtual stent with computer-aided-design with respect to an embodiment as shown in FIG. above. Once again, the features of the invention shown relating to the modeling process which was set forth in detail above. It is further noted that use of this process in any number of body lumens is contemplated as within the scope of the instant teachings.

Turning now to FIG. 10, a photographic image of a cast made from a harvested carotid artery according to the teachings of the present invention and the process thereunder is further illustrative of the instant process, set forth above and claimed below. Adjustments made for ‘shrinkage’ of these vessels were made also, within the empirical protocal developed by the present inventor.

A bottom terminal portion of the present inventor's first discussed carotid stent embodiment 10, as also shown in FIG. 2 and FIG. 5 is shown at FIG. 11. Note that this figure shows a (stent scaffolding geometry free) view of a ‘trumpet-like’ portion adjacent ovaloid opening 11 in a first embodiment of the present invention, including the three ordinal planes (X, Y, and Z) as reference points and parabolic curvilinear markers (A, B) defining the peripheral portions of the involved stent. Stent 10 has a lower end or an inlet 11 and an upper end 12 (not shown). Inlet 11 is found in the region of vessel bifurcation 3 and is shaped in ovaloid form in order not to cover the outlet of the external carotid artery 5 (previously shown).

Stent 10 is also ‘trumpet-like’ or shaped like a cone, wherein the radial diameter varies in the longitudinal course and becomes smaller proceeding from inlet 11 upper end 12, so that it is adapted to the course of internal carotid artery 4. Likewise, the geometry of stent 10 can be characterized as roughly parabolic, making reference to the three ordinal planes which are shown at each radius-based juncture where measurements are taken. The conical, parabolic, or ‘trumpet-shaped’ nature of stent 10 is noted as distinct from those generally tubular stents which are known and used conventionally.

Stent 10 has a tectonic structure, i.e., angles and curvatures in three-dimensional space adapted to the course of internal carotid artery 4. Stent 10 according to the invention is characterized by an anatomically corrected adaptive form, as further defined below within the claims that are appended hereto.

Referring now to FIG. 12, which shows a similar view of a scond embodiment of the present inventor's carotid version of his virtual stent (stent scaffolding geometry free), this view being of a ‘trumpet-like’ portion adjacent to the ovaloid opening. As discussed with respect to FIG. 11, in a second embodiment of the present invention, including the three ordinal planes (X, Y, and Z) as reference points and parabolic curvilinear markers (A, B) defining the peripheral portions of the involved stent.

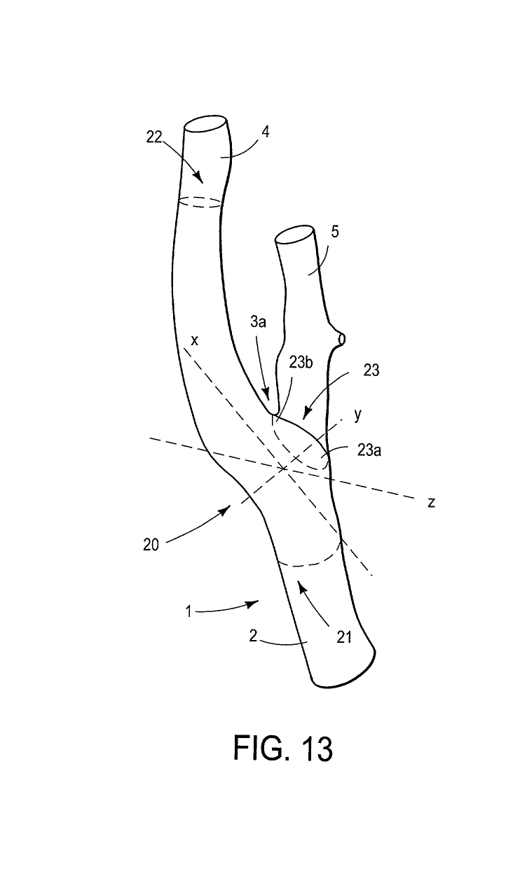

FIG. 13 shows schematically the location of the view shown in FIG. 12 relative to a second embodiment of the instant teachings as illustrated in the FIG. 3 series above. Referring to the figure, an outlined schematic showing the claimed peripheral portions of stent 20 according to the present invention is shown. Please note that, as shown, it is possible to use patterns, portions, or desired aspects of any known or developed stent geometry or scaffolding structure to satisfy the desired tectonic structure. A ‘tapestry’ matching any particular endoluminal body lumen which has been modeled mya be employed using either known or developed aspects, portions or types of scaffolding geometry.

According to the instant teachings, there is disclosed generation of novel enhanced optimized, or customized stents for any desired body lumens. By targeting a desired luminal surface to be mapped, it is possible to garner a required number of data points and used a computer-aided design to offer an appropriate series of tectonics for the required stent.

The present inventor has reduced this to practice by using the example of the heretofore inaccessible carotid arterial bifurcation. In lieu of the present inventor's casting steps, data that is known or previously stored for patients is also used, arrayed, and plotted to come up with individuated stent sections to match the required internally mapped luminla surface.

Returning to the carotid bifurcartion example shown in FIG. 13, stent 20 is also shaped like a cone, as is stent 10, wherein the radial diameter varies in the longitudinal course and becomes smaller from lower end 21 to upper end 22, so that it is adapted to the course of internal carotid artery. Stent 20 likewise has a tectonic structure, i.e., it has angles and curvatures in three-dimensional space adapted to the course of internal carotid artery 4, or it is “trumpet-shaped” or roughly parabolic. Stent 20 according to the invention is characterized by an anatomically correct adaptive form, whereby it is differentiable from the conventional tubular stenting devices which make up the majority of the prior art disclosures.

Stent 10 or 20 is comprised of a grid network, which can be formed of metal and/or plastic. The material may also be bioresorbable. The grid network may be introduced in the desired from by a balloon catheter, or it may have a memory effect, so that it is converted to the desired from automatically by the action of body heat. Due to the tectonic structure of the grid frame, stent 10, 20 can reconstruct the curve-shaped course of the internal carotid artery in three-dimensional space. Bending at a sharp angle is thus avoided.

Since individuated sections may be combined to create the device once a virtual stent has been modeled, substantial progress in science and the useful arts is believed to have been achieved as defined in the claims which are appended hereto, which are intended to be illustrative rather than limiting of the teachings of the present invention which necessarily must be so de-limited.

TABLE 1
Results of carotid stenting in literature 1996-2000

| | | | | | Apoplexy |
|---|---|---|---|---|---|
| | | Proportion | Apoplexy | | rate/ |
| Author | n | Stage I | rate | Lethality | Lethality |
| Roubin 1996 | 152 | 37% | 5.9% | 0.7% | 6.6% |
| Diethrich 1996 | 110 | 72% | 6.4% | 1.8% | 7.3% |
| Yadav 1997 | 126 | 42% | 7.1% | 0.8% | 7.0% |
| Wholey 1997 | 108 | 44% | 3.7% | 0.9% | 5.6% |
| Jordan 1998 | 268 | 63% | 6.6% | 1.1% | 9.7% |
| Henry 1998 | 173 | 65% | 2.9% | — | 2.9% |
| Mathias 1999 | 633 | 30% | 2.7% | 0.3% | unknown |
| Wholey | 2048 | no data | 4.4% | 1.4% | no data |
| (1998 survey) | | | | | |
| Wholey | 4757/ | no data | 4.21% | 0.86% | 5.07% |
| (2000 survey) | 5210 | | | | |

[image] TABLE 3
Area calculation at measuring points C1 and C2 of the
ACC and at measuring points I1 and I2 of the ACI

| | | | | | | | Rank sum test | |
|---|---|---|---|---|---|---|---|---|
| | | Mean | Standard- | | 1st quartile, | Minimum, | mean p-value | |
| | | value | deviation | Median | 3rd quartile | maximum | rank | |
| | Number | [mm²] | [mm²] | [mm²] | [mm²] | [mm²] | [mm²] | |
| AC1 | | | | | | | | |
| All | 62 | 30 1 | 9.06 | 28.30 | 23.70, 33.55 | 15.29, 59.94 | | |
| Men | 35 | 32 05 | 10.21 | 28.51 | 23.70, 39.84 | 18.35, 59.94 | 34.31 | 0.16 |
| Women | 27 | 27.59 | 6.66 | 28.10 | 22.60, 31.24 | 15.29, 44.18 | 27.85 | |
| With ASKL | 56 | 30 16 | 8.97 | 28 93 | 23.70, 33.55 | 16.78, 59.94 | 31.61 | 0.90 |
| Without | 6 | 29.68 | 10.73 | 28.09 | 22.60, 39.84 | 15.29, 44.18 | 30.50 | |
| ASKL | | | | | | | | |
| AC2 | | | | | | | | |
| All | 59 | 26.24 | 8.52 | 24.68 | 20.35, 29.35 | 11.76, 60.10 | | |
| Men | 33 | 27.11 | 9.09 | 25.21 | 20.69, 29.78 | 16 04, 60.10 | 31.55 | 0.44 |
| Women | 26 | 25.13 | 7.77 | 24.27 | 19.80, 28.95 | 11.76, 45.70 | 28.04 | |
| With ASKL | 53 | 26 58 | 8.39 | 24.80 | 20.92, 29.31 | 14 24, 60.10 | 30 91 | 0.23 |
| Without | 6 | 23.22 | 9 90 | 19.52 | 18.61, 30 80 | 11.76, 39.09 | 22 0 | |
| ASKL | | | | | | | | |
| AI1 | | | | | | | | |
| All | 63 | 29.46 | 10.48 | 27.94 | 21.10, 37.50 | 9 34, 53 05 | | |
| Men | 36 | 31.90 | 10.44 | 33.26 | 23.26, 39.03 | 12.25, 53.05 | 36 36 | 0 03 |
| Women | 27 | 26.21 | 9.78 | 25.25 | 21.02, 29.16 | 9.34, 51.98 | 26 19 | |
| With ASKL | 57 | 28.98 | 10.64 | 27.42 | 21.10, 36.10 | 9.34, 53.05 | 31.04 | 0 20 |
| Without | 6 | 34.07 | 7.98 | 36.23 | 29.16, 37.89 | 21.02, 43.91 | 41.17 | |
| ASKL | | | | | | | | |
| AI2 | | | | | | | | |
| All | 60 | 15 13 | 4.36 | 14.64 | 12.70, 17.42 | 5.86, 26.36 | | |
| Men | 35 | 15.30 | 3.82 | 15.42 | 12.96, 17.67 | 5.86, 26.36 | 31.94 | 0.45 |
| Women | 25 | 14.89 | 5.09 | 14.01 | 11.29, 16.78 | 7.05, 24.08 | 28.48 | |
| With ASKL | 54 | 14 49 | 3.86 | 14.04 | 12.27, 16.78 | 5.86, 24.08 | 28.39 | 0.005 |
| Without | 6 | 20.94 | 4.65 | 22.48 | 16 14, 23.80 | 14 41, 26.36 | 49.50 | |
| ASKL | | | | | | | | |

TABLE 4
Area calculation at measuring point E1 (ACE outlet)
and at measuring point B (carotid bulbus)

| | | | | | | | Rank sum test | |
|---|---|---|---|---|---|---|---|---|
| | | Mean | Standard- | | 1st quartile, | Minimum, | mean p-value | |
| | | value | deviation | Median | 3rd quartile | maximum | rank | |
| | Number | [mm²] | [mm²] | [mm²] | [mm²] | [mm²] | [mm²] | |
| AE | | | | | | | | |
| All | 62 | 17.75 | 7.69 | 16.90 | 11.75, 20.41 | 5.43, 39.44 | | |
| Men | 35 | 19.81 | 8 96 | 18.23 | 11.56, 28.53 | 7.48, 39.44 | 34.91 | 0 09 |
| Women | 27 | 15.07 | 4.51 | 14.92 | 11.75, 19.43 | 5 43, 22.84 | 27.07 | |
| With ASKL | 56 | 17.64 | 7.96 | 16.35 | 11.72, 20.12 | 5.43, 39.44 | 30.71 | 0.30 |
| Without | 6 | 18 75 | 4 70 | 19 44 | 17.67, 22.84 | 10.25, 22 84 | 38.83 | |
| ASKL | | | | | | | | |
| AB | | | | | | | | |
| All | 62 | 53 15 | 18.12 | 50.50 | 40.34, 65.70 | 20.73, 97.20 | | |
| Men | 35 | 57 70 | 18 29 | 56 79 | 46.73, 68.67 | 20.73, 97.20 | 36,03 | 0 02 |
| Women | 27 | 47.27 | 16 41 | 43 69 | 34.29, 56.33 | 21.94, 83.02 | 25 63 | |
| With ASKL | 56 | 52 62 | 18 49 | 49.92 | 37.21, 65.68 | 20.73, 97.20 | 30.80 | 0.36 |
| Without | 6 | 58 12 | 14.56 | 52.02 | 50.30, 67.68 | 43.69, 83.02 | 38.0 | |
| ASKL | | | | | | | | |

TABLE 5
Angular sum of αi (ACI outlet angle) and αe (ACE outlet angle)
respectively calculated to the mainstream direction of the ACC

| | | | | | | | Rank sum test | |
|---|---|---|---|---|---|---|---|---|
| | | Mean | Standard- | | 1st quartile, | Minimum, | mean p-value | |
| | | value | deviation | Median | 3rd quartile | maximum | rank | |
| | Number | [°] | [°] | [°] | [°] | [°] | [°] | |
| ANGULAR | | | | | | | | |
| SUM | | | | | | | | |
| All | 61 | 44 85 | 16 70 | 45 0 | 34.5, 56.0 | 13.0, 78.0 | | |
| Men | 34 | 45 69 | 17.60 | 46.0 | 35.0, 60.0 | 14.0, 78.0 | 31.78 | 0.71 |
| Women | 27 | 43 80 | 15.75 | 45.0 | 28.0, 54.0 | 13.0, 77.0 | 30.02 | |
| With ASKL | 55 | 44.95 | 17.47 | 45.03 | 33 0, 60.0 | 13.0, 78.0 | 31.15 | 0.85 |
| Without | 6 | 43 92 | 7.18 | 45.5 | 36.5, 48.0 | 34.5, 53.5 | 29.58 | |
| ASKL | | | | | | | | |

TABLE 6
Calculation of the difference in area between
the measuring points I1 and I2 on the ACI

| | | | | | | | Rank sum test | |
|---|---|---|---|---|---|---|---|---|
| | | Mean | Standard- | | 1st quartile, | Minimum, | mean p-value | |
| | | value | deviation | Median | 3rd quartile | maximum | rank | |
| | Number | [mm²] | [mm²] | [mm²] | [mm²] | [mm²] | [mm²] | |
| AREA = | | | | | | | | |
| (AI1-A12) | | | | | | | | |
| All | 60 | 14.49 | 8.98 | 12.95 | 6.95, 21.54 | 0.50, 38.69 | | |
| Men | 35 | 16.61 | 9.74 | 17.60 | 6.94, 23.84 | 1.32, 38.69 | 34.20 | 0.053 |
| Women | 25 | 11 53 | 6.95 | 9.87 | 6.96, 15.69 | 0.50, 31.41 | 25.32 | |
| With ASKL | 54 | 14.64 | 9.24 | 13.85 | 6.94, 22.14 | 0.50, 38.69 | 30.69 | 0.81 |
| Without | 6 | 13 13 | 6.62 | 11.34 | 6.96, 21.16 | 6.61, 21.37 | 28.83 | |
| ASKL | | | | | | | | |

TABLE 7
Curvature at the proximal ACI outlet

| | | | | | | | Rank sum test | |
|---|---|---|---|---|---|---|---|---|
| | | Mean | Standard- | | 1st quartile, | Minimum, | mean p-value | |
| | | value | deviation | Median | 3rd quartile | maximum | rank | |
| | Number | [mm] | [mm] | [mm] | [mm] | [mm] | [mm] | |
| CURVATURE | | | | | | | | |
| All patients | 60 | 0 082 | 0 103 | 0.076 | 0.027, 0.153 | −0.233, 0.306 | | |
| Men | 33 | 0 096 | 0.091 | 0.079 | 0.042, 0.0161 | −0.066, 0.301 | 32.15 | 0.42 |
| Women | 27 | 0.064 | 0 116 | 0.073 | 0.023, 0.138 | −0.233, 0.253 | 28.48 | |
| With ASKL | 54 | 0.078 | 0.106 | 0.073 | 0.024, 0.152 | −0.233, 0.306 | 29.61 | 0.24 |
| Without ASKL | 6 | 0 124 | 0 061 | 0.130 | 0.073, 0.167 | 0.037, 0.205 | 38.50 | |
| CURVATURE | | | | | | | | |
| OF ABs | | | | | | | | |
| All | 60 | 0.108 | 0.075 | 0 078 | 0.046, 0 164 | 0.015, 0.306 | | |
| Men | 33 | 0 108 | 0.075 | 0.079 | 0.058, 0 161 | 0.015, 0.306 | 30.67 | 0.94 |
| Women | 27 | 0 107 | 0.077 | 0.078 | 0.037, 0.171 | 0.017, 0.253 | 30.30 | |
| With ASKL | 54 | 0.106 | 0.077 | 0.078 | 0.041, 0.161 | 0.015, 0.306 | 29.94 | 0.47 |
| Without ASKI | 6 | 0 124 | 0 061 | 0.130 | 0.073, 0.167 | 0.037, 0.205 | 35.50 | |

TABLE 8
Maximum and minimum diameters at the
measuring points on the ACC, ACI and ACE

| | | Mean | Standard- | Me- | 1st quartile, | Minimum, |
|---|---|---|---|---|---|---|
| | Num- | value | deviation | dian | 3rd quartile | maximum |
| | ber | [mm] | [mm] | [mm] | [mm] | [mm] |
| DC1 min | 62 | 5.51 | 0.85 | 5.36 | 4.82, 6.09 | 3.64, 7.74 |
| DC1 max | 62 | 6.86 | 1.18 | 6.76 | 6.0, 7.56 | 4.04, 10.12 |
| DC2 min | 59 | 5.16 | 0.85 | 4.95 | 4.56, 5.7 | 3.42, 7.8 |
| DC2 max | 59 | 6.36 | 1.11 | 6.21 | 5.68, 6.96 | 3.83, 9.81 |
| DI1 min | 63 | 5.62 | 1.20 | 5.62 | 4.6, 6.67 | 3.37, 8.09 |
| DI1 max | 63 | 6.49 | 1.18 | 6.55 | 5.86, 7.29 | 3.53, 8.79 |
| DI2 min | 60 | 4.04 | 0 66 | 3.99 | 3 59, 4.49 | 2.44, 5.48 |
| DI1 max | 60 | 4.69 | 0 70 | 4.66 | 4.3, 5.04 | 2.95, 6.44 |
| DE min | 62 | 4 27 | 1 01 | 4.14 | 3.48, 4.85 | 2.4, 6.48 |
| DE max | 62 | 5.08 | 1.06 | 4 97 | 4.35, 5.75 | 2.81, 7.75 |

TABLE 9
Angles αi (ACI outlet angle) and αe (ACE outlet angle)
respectively calculated to the mainstream direction of the ACC

| | | Mean | Standard- | | 1st quartile, | Minimum, |
|---|---|---|---|---|---|---|
| | Num- | value | deviation | Median | 3rd quartile | maximum |
| | ber | [°] | [°] | [°] | [°] | [°] |
| ALFAI | 60 | 23 42 | 12.23 | 21.75 | 14, 32 | 0, 58 |
| ALFAE | 60 | 22 18 | 12.45 | 20 | 13.5, 29 | 2, 67 |

TABLE 10
Calculated BMI in the entire collection

| | | Mean | Standard- | | 1st quartile, | Minimum, |
|---|---|---|---|---|---|---|
| | | value | deviation | Median | 3rd quartile | maximum |
| | Number | [kg/m²] | [kg/m²] | [kg/m²] | [kg/m²] | [kg/m²] |
| BMI | 55 | 24.45 | 4.99 | 23.8 | 14.75 | 38.06 |

TABLE 11
Correlation of BMI to the cross-section areas of the ACC,
ACI and ACE

| | | Spearmann | p-value |
|---|---|---|---|
| BMI/area | | rank correlation | hypothesis |
| correlation | Number | coefficient φ | φ = 0 |
| AC1 | 54 | −0.12 | 0.38 |
| AC2 | 51 | −0.06 | 0.65 |
| AI1 | 55 | −0.02 | 0.86 |
| AI2 | 53 | −0.22 | 0.10 |
| AE | 54 | 0.11 | 0.41 |
| AB | 54 | −0.06 | 0.62 |

What is claimed as new novel and unobvious and desired to be secured by the issuance of the instant U.S. Letters Patent is:

1. A Stent for implantation into the internal carotid artery, having a lower end and an upper end , wherein a radius measured from lower end to upper end decreases in value, wherein the stent has a ‘trumpet-like’ tectonic structure in the form of angles and curvatures adapted to, and tracking the course of the internal carotid artery and that in the region of the outlet of internal carotid artery, lower end is formed as an ovaloid opening.

2. A Stent for implantation into the internal carotid artery, with a lower end and an upper end, wherein the radius decreases from lower end to upper end, characterized by the fact that stent has a tectonic structure in the form of angles and curvatures adapted to the course of internal carotid artery (4), that lower end projects into the common carotid artery and that an ovaloid recess is provided in the region of the outlet of the external carotid artery.

3. Stent according to claim 1 formed as a hollow lattice frame, wherein the tectonic structure is formed by the lattice structure, further characterized in that it can be at least one of expanded automatically and balloon-expanded.

4. Stent according to claim 2 formed as a hollow lattice frame, wherein the tectonic structure is formed by the lattice structure, further characterized in that it can be at least one of expanded automatically and balloon-expanded.

5. Stent according to claim 3 further comprising at least a lattice frame which further comprises either an entire tube, individuated sections, zones, regions or segments which are at least one of bent, braided, knitted, and stamped from a tube.

6. Stent according to claim 4 further comprising at least a lattice frame which further comprises either an entire tube, individuated sections, zones, regions or segments which are at least one of bent, braided, knitted, and stamped from a tube.

7. Stent according to claim 5 further comprised of at least one of a coated, ensheathed, sandwiched and admixted material which impacts thrombogenecity.

8. Stent according to claim 6 further comprised of at least one of a coated, ensheathed, sandwiched and admixted material which impacts thrombogenecity.

9. Stent according to claim 5 further comprised comprised of a bioresorbable material or having a bioresorbable coating or sheathing.

10. Stent according to claim 6 further comprised comprised of a bioresorbable material or having a bioresorbable coating or sheathing.

11. Stent as defined by claim 1, further comprising a geometric architecture optimized by computer-aided-design within preset parameters specific to a human carotid artery.

12. Stent as defined by claim 2, further comprising a geometric architecture optimized by computer-aided-design within preset parameters specific to a human carotid artery.

13. Stent as defined by claim 3, further comprising a geometric architecture optimized by computer-aided-design within preset parameters specific to a human carotid artery.

14. Stent as defined by claim 4, further comprising a geometric architecture optimized by computer-aided-design within preset parameters specific to a human carotid artery.

15. Stent as defined by claim 5, further comprising a geometric architecture optimized by computer-aided-design within preset parameters specific to a human carotid artery.

16. Stent as defined by claim 6, further comprising a geometric architecture optimized by computer-aided-design within preset parameters specific to a human carotid artery.

17. Stent as defined by claim 7, further comprising a geometric architecture optimized by computer-aided-design within preset parameters specific to a human carotid artery.

18. Stent as defined by claim 8, further comprising a geometric architecture optimized by computer-aided-design within preset parameters specific to a human carotid artery.

19. Stent as defined by claim 9, further comprising a geometric architecture optimized by computer-aided-design within preset parameters specific to a human carotid artery.

20. Stent as defined by claim 10, further comprising a geometric architecture optimized by computer-aided-design within preset parameters specific to a human carotid artery.

21. In a stenting apparatus configured to correspond to the endoluminal surface of a carotid artery having a tectonic structure in the form of angles and curvatures following the course of a patient's internal carotid artery and having an ovaloid aperture communicating with the external carotid artery, the improvement which comprises a trumpet-like tapered section from proximal to distal ends, whereby the spatial orientation of the stent defines a substantially hyperbolic section adjacent said ovaloid aperture.

22. Stenting Apparatus as defined in claim 8, said substantially hyberbolic section is of a form:
$$y = \pm \frac{b}{a} \sqrt{{x}^{2} - {a}^{2}}$$
in which:
x and y are the principal axes of said hyperbola;
a=the distance along the x-axis from the origin to the point at which the hyperbola intersects the x-axis; and b=the distance in a direction parallel to the y-axis from the point at which the hyperbola intersects the x-axis to an asymptote of the hyperbola.

23. Stenting Apparatus as defined in claim 22, whereby a and b comprise approximately equal distances.

24. A Process for generating a virtual stent, comprising the steps of:
targeting a luminal surface to be mapped;
capturing a non-contact picture of the surface data of the luminal surface to be mapped;
generating a multiplicity of three-dimensional measuring points;
arraying said multiplicity of three-dimensional measuring points within a predetermined lattice structure defining a tectonic structure in the form of angles and curvatures adapted to the course of the mapped luminal surface;
providing individuated or otherwise customized sections of geometric scaffolding structure corresponding to the portions arrayed in the lattice by three-dimensional computer modeling to make a tectonic structure for a stent.

25. Process as defined in claim 24, further comprising:
creating a endoluminal stenting device based upon the resulting virtual stent.

26. Process of claim 23, where the mapping step is done by at least one measurement method selected from the group consisting of:
3-Dimensional colour duplex sonography;
laser mapping;
utlrasound techniques;
x-ray based viewing;
endoscopic data point gathering;
physical data point generation;
MR angiography; and
Spiral CT scanning.

27. Process of claim 24, where the mapping step is done by an internal luminal measurement method selected from the group consisting of:
3-Dimensional colour duplex sonography;
laser mapping;
utlrasound techniques;
x-ray based viewing;
endoscopic data point gathering;
physical data point generation;
MR angiography; and
Spiral CT scanning.

28. Process of claim 24, wherein the multiplicity of three dimensional measuring points ranges between at least about 50 and approximately 500,000.

29. A product, produced by the process of claim 24.

30. A product, produced by the process of claim 25.

31. A product, produced by the process of claim 26.

32. A product, produced by the process of claim 27.

33. A process according to claim 27, wherein the luminal surface mapped is arterial.

34. A process according to claim 27, wherein the luminal surface mapped is a carotid artery.

35. A process according to claim 27, wherein the luminal surface mapped is a An aorta.

36. A process according to claim 27, wherein the luminal surface mapped is within the peripheral vasculature.

37. A process according to claim 27, wherein the luminal surface mapped is a cornary artery, sinus or cardiac chamber or lumen.

38. A process according to claim 27, wherein the lumen is within a human body.

38. diac space.
